(19) United States
(12) Patent Application Publication
(10) Pub. No.: US 2011/0136236 A1
(43) Pub. Date: Jun. 9, 2011

(54) GENETICALLY MODIFIED EUKARYOTIC CELLS

(75) Inventors: Nicole Borth, Vienna (AT); Ian Fraser Pryme, Bergen (NO); Beate Stern, Beate (NO)

(73) Assignee: UniTargeting Research AS, Bergen (NO)

(21) Appl. No.: 12/988,973

(22) PCT Filed: Apr. 21, 2009
(86) PCT No.: PCT/IB2009/005495

§ 371 Date: Feb. 15, 2011

Related U.S. Application Data
(62) Division of application No. 61/046,560, filed on Apr. 21, 2008.

(30) Foreign Application Priority Data
Apr. 21, 2008 (SE) 0800913-6

(51) Int. Cl. C12N 15/63; C12N 15/87; C07H 21/04; C12N 5/10; C12N 1/15

(52) U.S. Cl. 435/455; 435/468; 435/471; 536/23.7; 435/320.1; 435/325; 435/419; 435/254.11

(57) ABSTRACT

The invention relates to a method of producing genetically modified eukaryotic cells with optimised growth characteristics wherein a recombinant first nucleotide sequence has been integrated into a desired position in the genome. The sequence contains at least one gene encoding a plasma membrane protein with either toxin-receptor or toxic properties and allowing for surface expression based cell sorting to identify a suitable genomic integration locus. The invention also relates to a second exogenous nucleotide sequence containing at least one protein of interest as well as a vector, which aids in the site specific exchange of the first with the second nucleotide sequence. Efficient exchange is achieved by a stringent selection strategy that will kill all cells still expressing the plasma membrane protein.

### FIELD OF INVENTION

The present invention relates to an improved process for the generation of genetically modified eukaryotic cells which, for example, accelerates clonal cell line production. The invention also relates to the use of said cell line(s).

### BACKGROUND OF INVENTION

Technology for expressing recombinant proteins (most of them are to be used as pharmaceuticals, e.g. human growth factors, antibodies, antibody-derived molecules, hormones, blood coagulation factors and cytokines) in mammalian cell factories is well established (Wurm 2004). Mammalian cells have been found to be required for the production of complex proteins to be used therapeutically due to their ability to post-translationally modify, e.g. glycosylate, recombinant proteins. Since they are for human use there is a requirement for high quality with regard to purity, optimal activity, functionality and stability. Furthermore, it is extremely important that recombinant proteins have no immunogenic effect in humans, which is also affected by their glycosylation pattern.

Treatment of humans with therapeutic proteins often requires the administration of large doses over a relatively long period of time. The increasing number of therapeutic protein drugs becoming accessible in the industrial pipeline is currently creating a substantial bottleneck as far as production efficiency is concerned. Since mammalian cell factory systems exhibit relatively ineffective production yields there is a considerable scope for their improvement.

A major challenge is the efficient selection of high-producing cells from a mixed population. This selection process can exceed six months. A reduction of the time period required to optimise clonal cell line generation is an area that offers great potential. Cell lines selected for high production rates, possessing high growth potential in large-volume fermenters, and stably expressing high amounts of product in a cost-effective manner are thus an extremely important target (Browne et al. 2007).

To be able to select and maintain the gene of interest in the transfected mammalian cell, selectable markers are often used, genetically linked to this gene encoding the protein of interest. Selectable drug resistance markers such as neomycin phosphotransferase or hygromycin B phosphotransferase are effective to obtain stable transfectants with the recombinant DNA integrated in the host genome. In addition to antibiotic selection, an amplification strategy for isolating high-producing clones is normally used involving co-expression of an amplifiable gene, such as the genes expressing dihydrofolate reductase (DHFR) (Alt et al. 1978) or glutamine synthetase (GS) (Cockett et al. 1990), requiring the addition of selection drugs such as methotrexate (MTX) or methionine sulphoximine (MSX), respectively. The disadvantages of growing cells in medium containing such drugs are that they are known to reduce growth rates, are toxic and expensive. Furthermore, the cells are required to synthesise the product of one or more other recombinant genes as well as the protein of interest. This will inevitably entail a considerable waste of energy on behalf of the cell.

With respect to amplification there are a number of additional problems: (i) in spite of the proximity of the gene for the selection marker and the gene of interest which would usually result in integration within the same site of the cellular genome there is not always a correlation between their expression; (ii) since the copy number of the genes is amplified, large regions on the host's chromosomes consist of exogenous DNA which increases the risk of chromosomal instability. Chromosomal rearrangements may result in the elimination of the repetitive stretches of DNA and methylation of transgenes present in tandem repeats in gene silencing. This might occur after many cell generations, even when the selected cell line is already in a highly productive phase of the protein of interest; (iii) a further problem is that the amplification in itself is a mutagenic process which will not only increase the gene copy number, but may also lead to changes in cell behaviour and metabolism. Due to the high mutation rate caused by transfection and amplification, even pre-adapted and pre-optimised host cell lines may change behaviour significantly during the screening process; (iv) since amplification is performed step-wise, several rounds of subcloning and screening are required, which is extremely time consuming; (v) finally there appears to be a limited amount of amplification that can be achieved due to the development of drug resistance.

When a transgene is transfected into a cell and integrated into the host genome, efficient expression of the gene is highly dependent on the site of integration. Even with repeated rounds of cloning and in the presence of selective pressure a homogeneous cell line does not result. Methods to circumvent this problem include epigenic gene-regulatory approaches exploiting specific cis-acting DNA elements (e.g ubiquitous chromatin opening elements, UCOEs, or matrix associated regions, MARs) in the transgene construct conveying high expression levels (Kwaks et al. 2006), or the targeting of sites in the host genome with high gene-expression potential by using retroviral vectors and site-specific cassette replacement methodology (Wirth et al. 2007). Disadvantages of these systems include, in the former case, the lack of knowledge regarding the molecular mechanisms involved and thus their unpredictability, and, in the latter case, regulatory concerns. Another focus addressing heterogeneity has been directed to high-throughput screening technology, including automated systems, which increase the chance of finding suitable cell lines by screening much larger cell populations in a shorter time span (Browne et al. 2007). Being based on the above-mentioned traditional methods, these systems, however, do not represent any significant development in the field of high-producer cell selection. The screening process, analysis of cell growth characteristics as well as confirmation of the stability of expression of the selected clones are still required for each new protein to be produced.

This invention discloses a novel selection system as well as a novel method to generate genetically modified eukaryotic cells such as producer cell lines with optimised properties with respect to productivity and growth behaviour in less than three months. A major aspect of the present invention is that it results in the creation of a “core cell line” with optimal growth properties and a defined chromosomal locus for maximal expression generated in a single round of transfection, selection, screening and analysis. This cell line may serve as a basis for the rapid generation of high-producer cells, for production of any protein of interest after a second transfection step and single-copy targeted integration. The new approach thus avoids the necessity of repeating the cumbersome selection, screening and analysis process for each new protein of interest, meaning that the time-consuming pitfalls discussed above can be effectively avoided. Further, it requires neither gene amplification nor high-level production of a superfluous selection protein as in the producer cell line the genes encoding the selection markers are replaced by the gene encoding the protein of interest. Finally, the approach is regulatory-friendly and adaptable to any eukaryotic host cell line, thus providing an attractive universal solution for high-yield recombinant protein production.

### SUMMARY OF THE INVENTION

The invention relates to a method of producing genetically modified eukaryotic cells or cell lines with optimised growth characteristics and wherein a single copy or few copies of a recombinant first nucleotide sequence has/have been integrated into a desired position(s) in the genome. The sequence contains at least one gene encoding a plasma membrane protein with either toxin-receptor or toxic properties and allowing for surface-expression based cell sorting to identify a suitable genomic integration locus. The invention also relates to a second exogenous nucleotide sequence containing at least one gene encoding at least one protein of interest as well a vector(s), which aids in the site-specific exchange of the first with the second nucleotide sequence. Efficient exchange is achieved by a stringent selection strategy that will invariably kill all cells still expressing the plasma membrane protein, since cell survival in the presence of a toxin or absence of a protective agent, respectively, in the cell growth medium is only possible after gene exchange. Thus, the invention comprises a two-step method in which the first step is to generate a reusable core cell line with a tagged integration site for stable transgene expression and with optimized growth characteristics. In the second step a producer cell line is made from the core cell line by replacing the gene(s) encoding the selectable plasma membrane protein marker with the gene(s) encoding any protein of interest. The novelty of this approach lies mainly in the ease and efficiency with which cells are pre-optimised for production and with which stably expressing producer cell lines can be generated from these cells for any protein of interest. It also lies in the fact that the producer cell line is engineered to only produce the protein(s) of interest, as the replacement of the selectable marker gene(s) is the basis for cell line selection. This inverts selection from being dependent on the presence of a selectable marker in standard systems to its absence in this invention. Intriguingly, the approach is adaptable to any eukaryotic host cell line and has a broad application range, including but not limited to (a) time-saving, high-yield and stable production of therapeutic proteins from cell culture systems, (b) comparable expression studies using e.g. various vectors or vector libraries, and (c) the generation of genetically modified cells for live cell therapy.

### BRIEF DESCRIPTION OF THE DRAWINGS

FIG. 1 is a schematic overview of the principle behind the selection method described in this invention. It is exemplified for the case where a plasma membrane protein is used with toxin-receptor properties. RMCE, recombinase-mediated cassette exchange.

FIG. 2 shows maps of plasmids representing or containing examples of the first nucleotide sequence (panels A, C, E) and the second nucleotide sequence (panels B, D, F) used to generate the core cell line and the producer cell line. Regarding the toxin receptor system, the plasmids are either based on CD11b/CD18 (panels A, B, E, F) or GC-C (panels C, D), and regarding the site-specific recombination system either on Flp/FRT (panels A, B, E, F) or Cre/lox (panel C, D). HC, antibody heavy chain; LC, antibody light chain.

FIG. 3 shows the toxin response of a mixed population of receptor-negative (40%) and receptor-positive (60%) cells. The receptor is the integrin protein CD18/CD11b and the toxic agent Adenylate cyclase.

FIG. 4 shows flow cytometric analysis of intracellular antibody in cell pools. CHO-S cells were either non-transfected (control, left panel) or transfected with pcDNA-select1 and subsequently with pcDNA-target1 and pcDNA-FlpE (right panel). Intracellular antibody amount (y axis) was plotted against cell size (x axis).

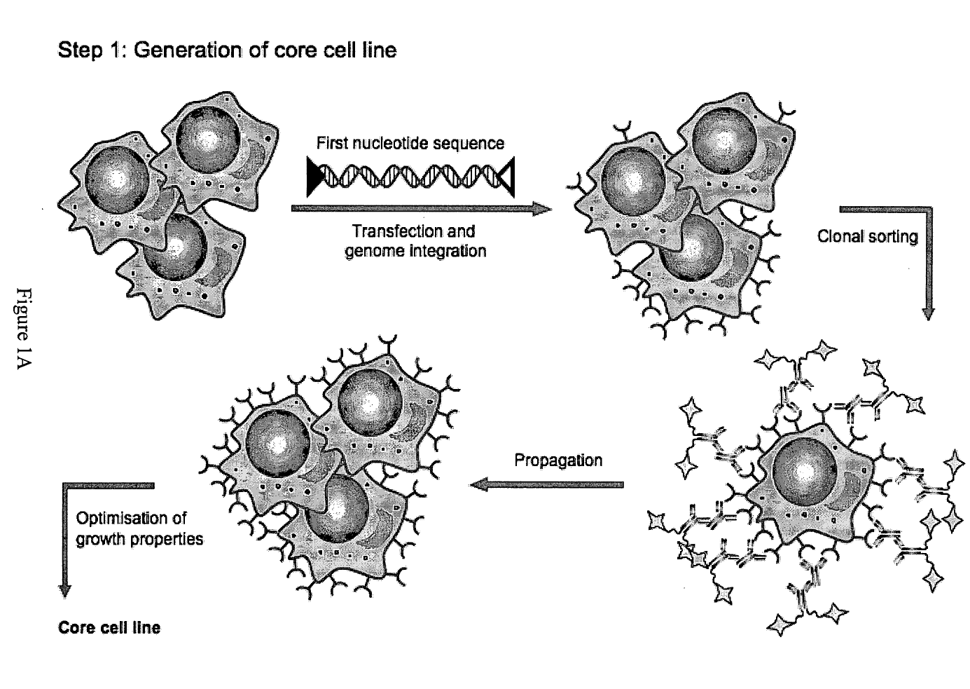

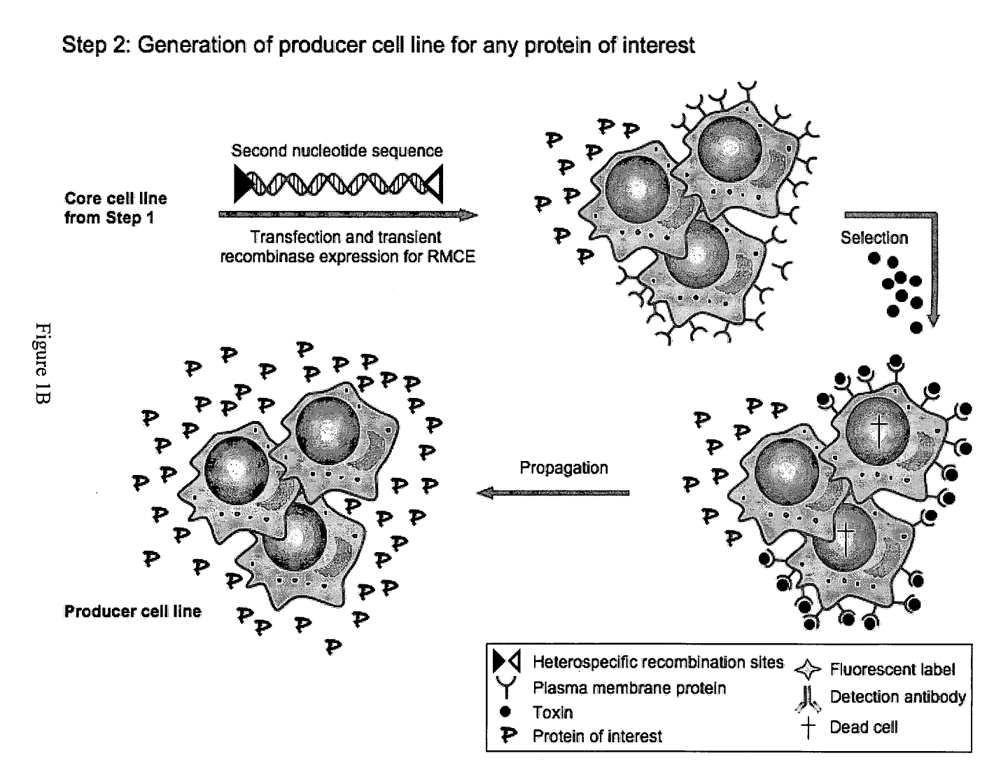

### DETAILED DESCRIPTION OF THE INVENTION

Unless defined otherwise, all technical and scientific terms used herein have the same meaning as commonly understood by those ordinarily skilled in the art to which the invention belongs.

For the purposes of the present invention, the following terms are defined below.

The term “nucleotide sequence” is intended to mean a sequence of two or more nucleotides. The nucleotide may be DNA, RNA as well as a mixture thereof, and of natural, semi-synthetic or synthetic origin.

The term “genetically modified eukaryotic cell” is intended to mean a eukaryotic cell containing at least one recombinant gene (transgene) that has been inserted into the genome of the eukaryotic cell.

The term “polypeptide” is intended to mean a peptide consisting of more than two amino acids.

The terms “5′UTR” and “3′UTR” are intended to mean the 5′ and 3′ untranslated regions on the mature mRNA flanking the coding sequence of a polypeptide.

The term “signal peptide” is intended to mean an N-terminal polypeptide, typically 15-30 amino acids long, targeting a polypeptide for translocation across the endoplasmic reticulum membrane in eukaryotic cells and cleaved off during the translocation process.

The term “control element” is intended to mean a nucleotide sequence involved in transcriptional and/or translational regulation of a gene contained on a vector, such as a plasmid or a DNA fragment, to be transfected into a eukaryotic cell or a nucleotide sequence involved in the replication of a plasmid. Examples include promoter, 5′UTR, 3′UTR, signal peptide coding region, poly(A) signal and replication origin.

The term “gene” is intended to mean a continuous nucleotide sequence constituting one transcription unit which is comprised of a coding sequence of a polypeptide and the corresponding transcriptional/translational control elements. The term “gene of interest” is intended to mean a gene encoding a “protein of interest” (see below).

The term “site-specific recombination sites” is intended to mean distinct short nucleotide sequences recognised by specific enzymes (site-specific recombinases) which catalyse precise DNA rearrangements. Two non-interacting heterospecific sites enable the possibility to replace genetic information located between them with other genetic information by the aid of the corresponding recombinase(s). The process, which is strictly unidirectional, is referred to in the literature as Recombinase-Mediated Cassette Exchange (RMCE).

The term “homologous recombination” is intended to mean DNA rearrangements occurring between two strands of DNA with similar sequences and mediated by the recombination machinery of a eukaryotic cell.

The term “transfection” is intended to mean the process by which a vector is inserted into the genome of a eukaryotic cell.

The term “protein of interest” is intended to mean any polypeptide encoded by a gene or any protein encoded by one or more genes, of which there is a need for obtaining an appropriate quantity for specific purposes and which is to be produced in a recombinant manner by cultivated eukaryotic cells.

The terms “5′ nucleotide sequence” and “3′ nucleotide sequence” are intended to mean distinct nucleotide sequences flanking and being part of a specified nucleotide sequence.

The invention relates to a new method for isolation of a genetically modified eukaryotic cell(s) comprising the steps of providing a eukaryotic cell(s) and at least one first nucleotide sequence, wherein said first nucleotide sequence comprises a 5′ nucleotide sequence and at least one gene encoding a plasma membrane protein, wherein said plasma membrane protein is a toxic protein or a toxin receptor and a 3′ nucleotide sequence, wherein said 5′ and 3′ nucleotide sequences allow for the exchange of the enclosed nucleotide sequence, transfecting said first nucleotide sequence into the genome of said eukaryotic cell(s), screening for a core cell(s) wherein said core cell(s) has at least one first nucleotide sequence integrated in the genome and expressing said at least one gene encoding a plasma membrane protein, propagating said core cell(s) and obtaining at least one core cell line.

In another aspect the invention relates to the generation of a producer cell line, wherein said method as defined above comprises the additional steps of: providing at least one second nucleotide sequence which comprises a 5′ nucleotide sequence and at least one gene encoding a protein(s) of interest and a 3′ nucleotide sequence, wherein said 5′ and 3′ nucleotide sequences are compatible to those present in the first nucleotide sequence, replacing said first nucleotide sequence in said at least one core cell line with said second nucleotide sequence, selecting for at least one producer cell wherein said producer cell(s) has at least one second nucleotide sequence integrated into the genome and expressing said at least one gene encoding a protein(s) of interest, propagating said producer cell(s) and obtaining at least one producer cell line. The producer cell line(s) may be used for the production of one or more proteins of interest, such as polypeptides and proteins that will be used for the development of pharmaceutical formulations.

Said eukaryotic cell(s) may be an animal cell, a plant cell, fungal or yeast cell. One example being that the animal cell is a mammalian cell. Other examples are that said mammalian cell is selected from the group consisting of primate-, monkey- and rodent-derived cells. Other examples are that said primate cell is of Homo sapiens or Pan troglodytes origin, said monkey cell is of Cercopithecus aethiops origin, and said rodent cell is of Cricetulus griseus, Mesocricetus auratus, Rattus norvegicus, Oryctolagus cuniculus or Mus musculus origin. Other examples are that said eukaryotic cell belongs to any of the cell line families CHO, NS0, 293, myeloma, NOS, COS, BHK, HeLa and PER.C6.

The first nucleotide sequence contains at least one gene encoding a plasma membrane protein functional in eukaryotic cells with either toxin-receptor or toxic properties. Cells bearing this recombinant protein on their surface are either sensitive to a specific toxic agent added to the cell growth medium or require a protective agent in the medium in order to survive. The plasma membrane protein thus acts as a selectable marker mediating cell death after application of a toxic agent and/or removal of a protective agent.

Examples of such plasma membrane proteins and their corresponding toxins or protective agents are listed in Table 1. The choice of the plasma membrane protein may depend on the cell line to be employed. Excluded are those plasma membrane proteins which are naturally expressed by the particular cell line. Further this cell line must not be sensitive to the corresponding toxins. In these cases, an option, however, would be to manipulate the cells such that they become deficient for said plasma membrane protein and/or toxin resistant. Further, the first nucleotide sequence may contain a gene encoding a drug resistance marker. Examples are antibiotic resistance genes such as neomycin phosphotransferase I, neomycin phosphotransferase II, hygromycin B phosphotransferase, blasticidin, blasticidin S deaminase, puromycin N-acetyl-transferase, bleomycin resistance gene.

Additionally the first nucleotide sequence as well as the second nucleotide sequence comprises compatible 5′ and 3′ nucleotide sequences that are target sites for a recombinase(s). One example is heterospecific recombination sites. Examples of such sites and their corresponding site-specific recombinases are given in Table 2. This enables a recombinase-mediated exchange of the first nucleotide sequence with a second nucleotide sequence comprising at least one gene encoding a protein(s) of interest. In this way the gene(s) encoding the selectable plasma membrane protein and optionally a drug resistance marker are removed and the gene(s) encoding the protein(s) of interest inserted. Instead of using a site-specific recombination system it is also possible to use other nucleotide exchange systems, such as a homologous recombination system. The recombinase may be encoded by a third nucleotide sequence and/or endogenously expressed by said eukaryotic cell(s).

TABLE 1
Examples of plasma membrane proteins with toxin-receptor
properties (A) and toxic properties (B) encoded by the first
nucleotide sequence and expressed in the core cell line.
A)

| Toxin receptor | Toxin | Origin | Function | Reference |
|---|---|---|---|---|
| Receptor-like | VacA | Helicobacter pylori | Vacuole | (Nakayama |
| protein tyrosine | | | formation, | et al. 2006) |
| phosphatase β | | | induction of | |
| (RPTPβ) | | | apoptosis | |
| Cell wall protein 2 | PMKT killer | Pichia membranifaciens | Forms non- | (Santos et al. |
| (Cwp2p) | toxin | | selective | 2007) |
| | | | channels leading | |
| | | | to loss of small | |
| | | | metabolites | |
| Guanylyl cyclase | Heat-stable | Escherichia coli | Elevation of | (Hasegawa |
| C (GC-C) | enterotoxin | | cGMP levels, | et al. 2005) |
| | (STₐ) | | interferes with | |
| | | | the cell's ion | |
| | | | homeostasis | |
| α_{M}β₂ integrin | Adenylate | Bordetella pertussis | Elevation of | (El-Azami- |
| (CD11b/CD18) | cyclase toxin | | cAMP levels, | El-Idrissi et |
| | (CyaA) | | interferes with | al. 2003) |
| | | | the cell's ion | |
| | | | homeostasis | |

B)

| | Protective | | | |
|---|---|---|---|---|
| Toxin | agent | Origin | Function | Reference |
| α-toxin | Polyethylene | Staphylococcus | Pore-formation | (Joubert et |
| | glycol (PEG) | aureus | in plasma | al. 2007) |
| | | | membrane | |

TABLE 2
Examples of site-specific recombination systems with heterospecific sites
flanking the first and second nucleotide sequence to enable their exchange
in producer cell line generation.

| Site specific | | | | |
|---|---|---|---|---|
| recombination | Heterospecific | | | |
| system | recombination sites | | Recombinase | Reference |
| Flp/FRT | FRT wt | F₃ | Flp | (Schlake et |
| | | | | al. 1994) |
| | FRT wt | F₅ | Flp | (Schlake et |
| | | | | al. 1994) |
| | FRT wt | F-5F | FlpE | (Ellermeier |
| | | | | et al. 2002; |
| | | | | Schucht et |
| | | | | al. 2006) |
| Cre/loxP | loxP wt | various mutant | Cre | (Saito and |
| | | loxP sites | | Tanaka) |
| | loxP wt | loxP511 | Cre | (Hoess et al. |
| | | | | 1986) |
| | loxP wt | m2 | Cre | (Langer et |
| | | | | al. 2002) |
| | loxP | loxP257 | Cre | (Wong et al. |
| | | | | 2005) |
| Flp/FRT and | loxP | FRT | Cre and Flp | (Lauth et al. |
| Cre/loxP | | | | 2002) |
| ΦC31/ | attP | attB | ΦC31 | (Thyagarajan |
| attP-attB | | | integrase | et al. 2001) |

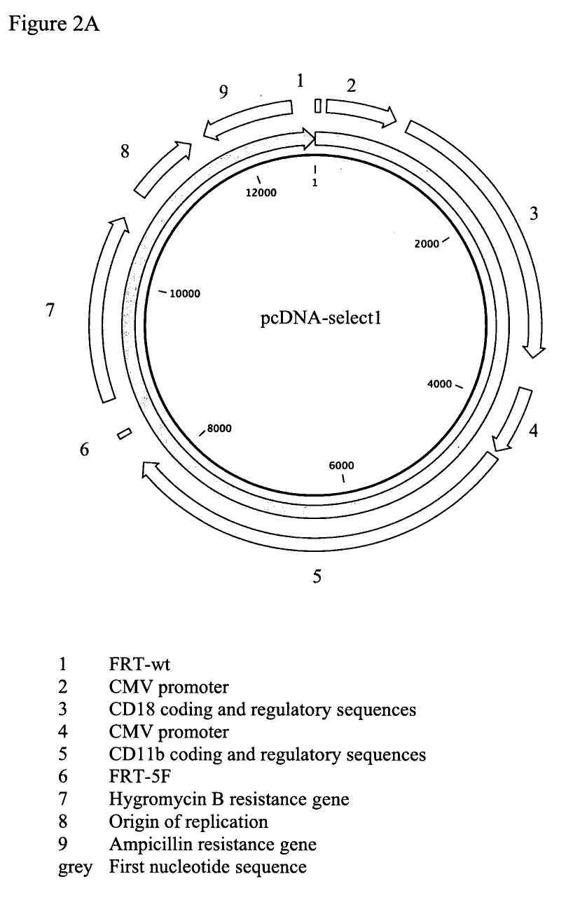

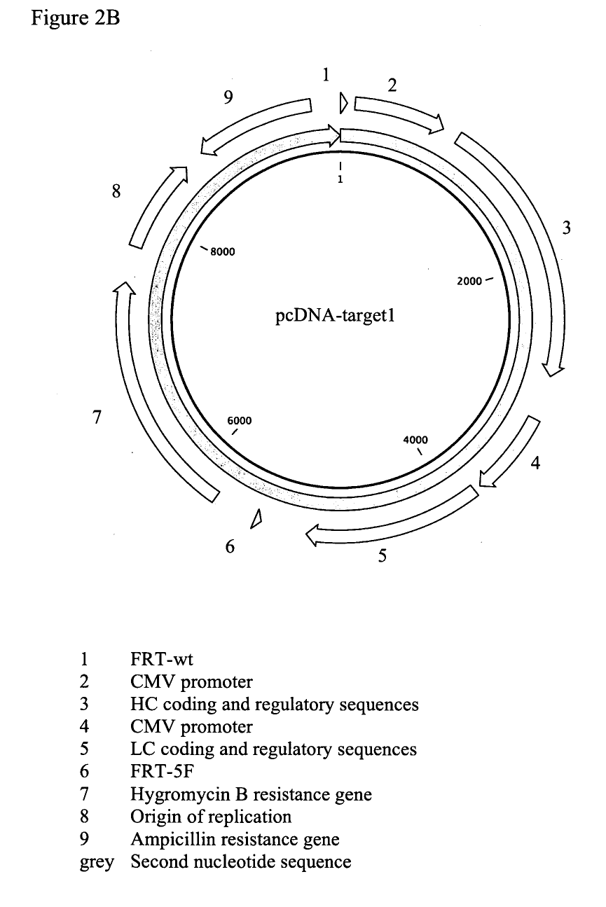

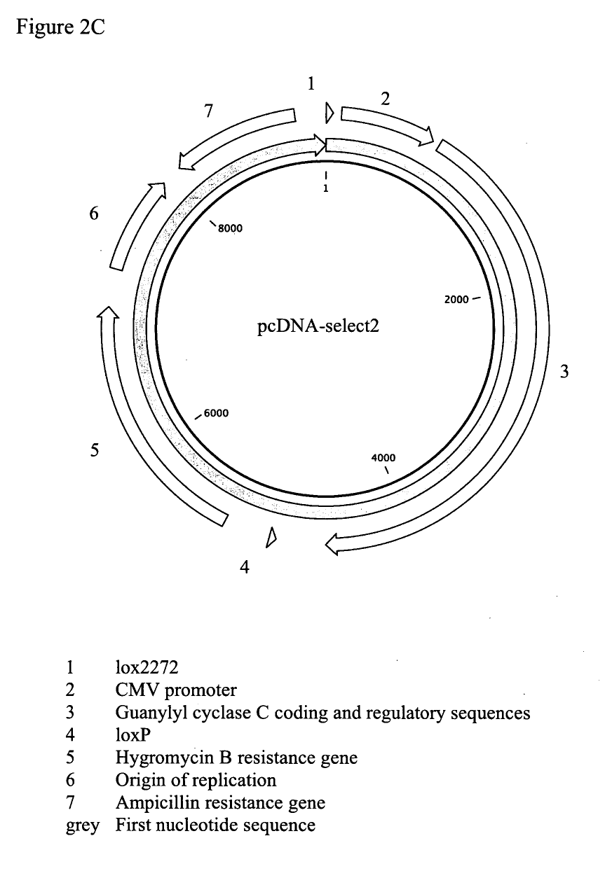

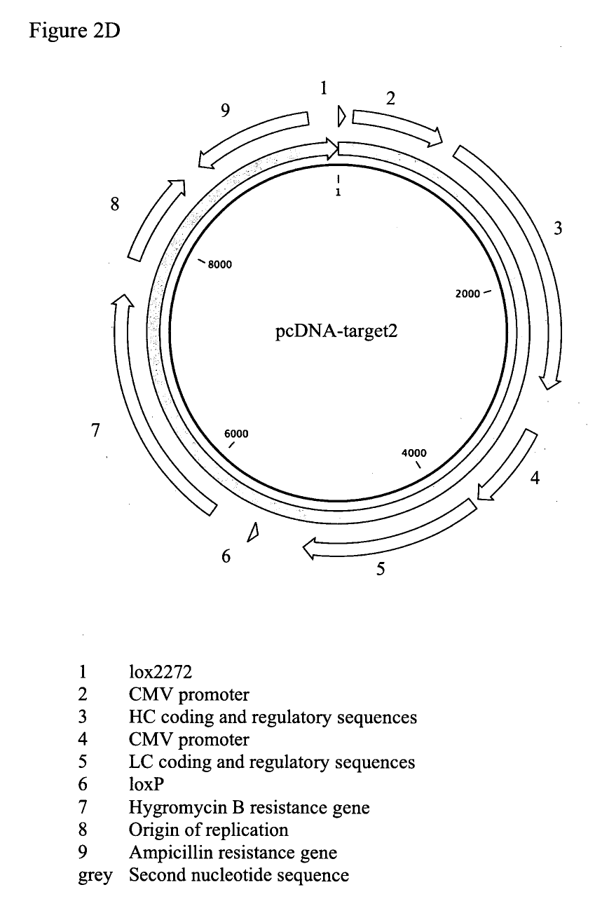

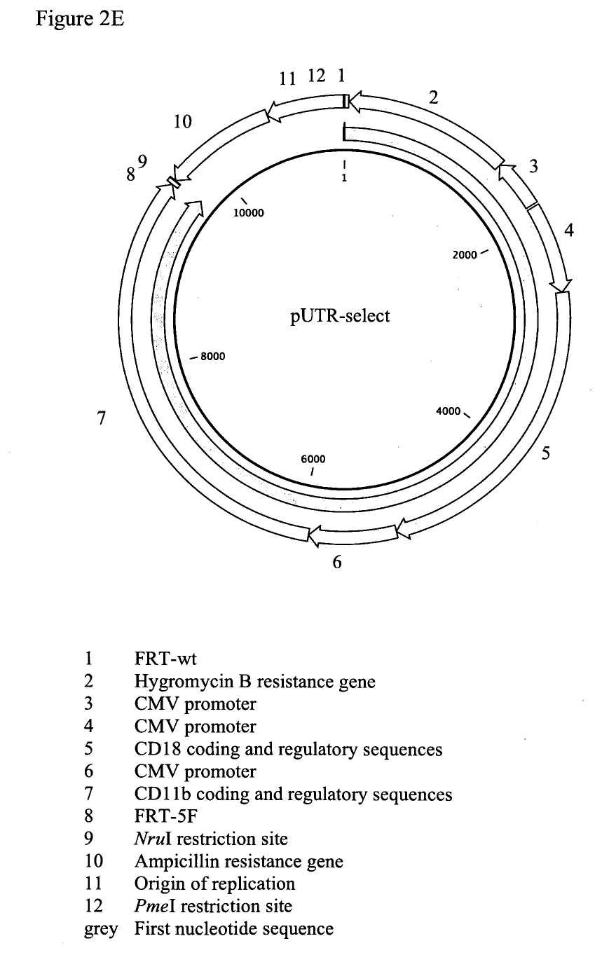

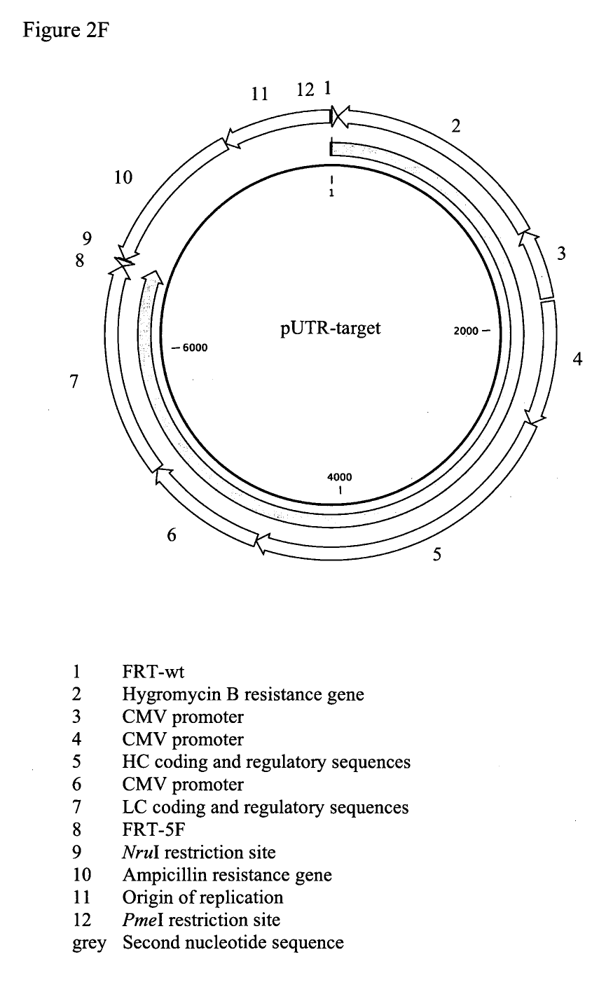

The first nucleotide sequence may be part of a vector, such as a plasmid, of which an examples are shown in FIG. 2A,C,E, which may be introduced into said eukaryotic cell(s) by transfection. Selection for stable transfectants with the gene(s) encoding the plasma membrane protein integrated into the cells' genome may be performed by use of a drug resistance marker or the plasma membrane protein. Since integration occur randomly, the transfectants obtained represent a heterogeneous population. In the individual cells of the population the first nucleotide sequence will reside in different chromosomal domains and a broad range of transcriptional activity will occur. One of various possible methodologies will then be used to identify the cell or cells that express the introduced gene(s) encoding the plasma membrane protein at a specific level. The level required will depend on the ultimate application to be implemented. However, in most cases it will be desirable to express the gene to as high a level as possible.

The identification and isolation of single cells can be performed using any suitable screening technique such as limited dilution cloning, flow cytometry and cell sorting as well as automated systems (Browne et al. 2007). As the gene(s) in the first nucleotide sequence encodes a plasma membrane protein containing an extracellular surface domain, using flow cytometry it is possible to sort for cells with specific expression levels by staining with antibodies recognizing this domain (Borth et al. 2000; Carroll et al. 2004). Each of the selected cells expressing the plasma membrane, protein to a desired level, mostly to the highest level in the cell population analysed, will then be grown to create a genetically modified eukaryotic cell line. As no amplification of the gene copy number is involved when generating these cell lines, the desired expression level will be achieved from a single gene copy or a few gene copies of the gene(s) encoding the plasma membrane protein. Southern blot analysis can be performed in order to detect cell lines with single copy integration and fluorescent in situ hybridization (FISH) to determine the chromosomal localisation of the integration site. Single copy integrations at loci not having known, negative position effects would increase the chance that long-term stability of gene expression will be achieved. Subsequently, the obtained subclones can be analysed for the stability of the level of expression of the plasma membrane protein over time.

The resulting isolated cell line(s) having stably integrated the first nucleotide sequence at a favourable position in the genome and displaying desired expression levels over many generations can be further subjected to cell line optimisation in order to identify exceptional cells with improved properties such as increased growth rate and final cell density, improved energy metabolism (through e.g. lower lactate production), high viability and apoptosis resistance, high genetic stability and good capacity to produce recombinant proteins. This can be achieved by using new methods of fluorescence-activated cell sorting (FACS) (Mattanovich et al. 2006) or any other appropriate method including metabolic engineering.

The resulting cell line(s), termed “core cell line(s)”, will serve as the basis for the generation of a producer cell line(s) for any protein of interest. Since the producer cell line(s) inherits the optimal growth and production properties from the particular core cell line generated for a specific application, cumbersome and time-consuming optimisation of each producer cell line is superfluous.

The invention thus also relates to the actual establishment of a producer cell line(s) wherein said method comprises the steps of: providing a second nucleotide sequence comprising at least one gene encoding a protein(s) of interest and 5′ and 3′ recombination sites compatible to those in said first nucleotide sequence, replacing said first nucleotide sequence in said core cell line with said second nucleotide sequence by recombination, selecting for at least one producer cell wherein said producer cell has at least one second nucleotide sequence integrated in the genome and expressing said at least one gene encoding a protein(s) of interest, propagating said at least one producer cell and obtaining at least one producer cell line. Said method will be performed by transfecting the core cell line(s) comprising a first nucleotide sequence comprising the gene(s) encoding the plasma membrane protein with a second nucleotide sequence comprising the gene(s) encoding the protein(s) of interest and said 5′ and 3′ nucleotide sequences which are compatible with those flanking and included in the first nucleotide sequence already integrated into the genome of the core cell line. The second nucleotide sequence may be part of a vector, such as a plasmid, of which an examples are shown in FIG. 2B,D,F. To ensure, if desirable, that the sequence does not contain any DNA of viral origin, a non-viral promoter could be used and recombination sites derived from e.g. yeast. The specific recombinase(s) required to mediate the exchange of the first with the second nucleotide sequence may be transiently introduced into the cells by any suitable method, e.g. by co-transfecting the cells with a third nucleotide sequence comprising the recombinase gene(s). After recovery of the cells and gradual depletion of the recombinase(s) during cell division, selective pressure will be applied. Depending on whether a core cell line has been used expressing a plasma membrane protein with toxin-receptor or toxic properties, the corresponding toxic agent will be added to the growth medium, or protective agent removed from the medium, which will kill all cells where complete replacement of the plasma membrane protein encoding gene(s) with the gene(s) encoding the protein(s) of interest has not taken place. The frequency of site-specific recombination in a chromosomal background is known to be low. However, as the selection strategy is extremely stringent, isolation of the producer cell(s) can readily be achieved.

The invention described here has several advantages over the conventional selection methods in current use:
(i) It allows for the identification of chromosomal regions in eukaryotic cells with desired transcriptional activity levels and high transcriptional stability.
(ii) It enables further optimisation of these cells with respect to their growth characteristics and genetic stability.
(iii) It does not involve gene amplification, but rather relies on single-copy targeted integration.
(iv) It is energy efficient, since the selection markers (plasma membrane protein and antibiotic resistance protein) are expressed only by the core cell line. The producer cell line is not required to waste energy on the high-level production of two or more proteins of which only one is the actual target.
(v) It provides a stringent selection strategy to achieve efficient incorporation of the gene(s) encoding the protein(s) of interest into a pre-defined chromosomal region.
(vi) It avoids the potentially disadvantageous effects of toxic agents on the producer cell line caused by long-term exposure. In fact, after the initial selection phase, the producer cell line is cultivated without any selective pressure.
(vii) It is regulatory-friendly, since only the nucleotide sequences needed for expression of the protein(s) of interest will remain in the producer cell line. In particular, any virus-derived components can be avoided.
(viii) It does not require repeated rounds of selection and screening due to a two-step strategy and the distinction between a core cell line and a producer cell line derived thereof. A producer cell line for any protein of interest can be obtained in a single cloning step resulting in a significant reduction in time.
(ix) It is adaptable to any host cell line and thus provides a universal solution.

It is important to emphasise that this invention, in contrast to a previously described selection method also using a two-step, though retrovirus-based approach (Coroadinha et al. 2006), exploits a plasma membrane protein as a marker. The membrane protein is synthesised in, and passes through, the same subcellular compartments as secreted proteins. This group of proteins, which includes antibodies, represents the main focus of interest in the biopharmaceutical industry to date. Cell lines initially screened for optimal production of a membrane protein can be expected to be better suited for the development of high-producing cell lines for secreted proteins than those where the selection is based on an intracellular, non-secreted protein. Further, due to the specific properties of the plasma membrane protein, this choice enables, for the first time, the exploitation of the same protein both for efficient cell screening and subsequent efficient selection. Selection is on the basis of the absence rather than the presence of a marker protein, thus avoiding the requirement for its production in addition to that of the protein(s) of interest. It also avoids having to add selective drugs during producer cell cultivation that may affect cell growth and viability. Finally, it provides for a selection system that is extremely stringent.

The invention described here may be used for many applications, including the following:
1. For the high-level production of any protein of interest, such as those manufactured in industrial production platforms. Examples of such proteins are human insulin, somatotropin, tissue plasminogen activator, α-interferon, β-interferon, γ-interferon, erythropoietin, granulocyte-stimulating factor, granulocyte-macrophage-stimulating factor, epidermal growth factor, factor VIIIc, factor IX, glucocerebrosidase, interleukin-2, interleukin-3, interleukin-4, stem cell factor, hormones, blood coagulation factors, cytokines as well as antibodies such as anti-CD3 antibody, CAMPATH-1H (anti-lymphocyte antibody), anti-endotoxin antibody and anti-tumour necrosis factor or any other antibody or antibody-derived molecules (e.g. Fab fragments, single chain antibodies, multivalent antibodies, antibody fusion proteins) designed for therapeutic use.
2. For the generation of genetically modified cells to be used in live cell therapy or tissue engineering. Examples where cell therapy treatment could be implemented are Fabry's disease, Niemann-Pick disease, Gaucher's disease, single cell anemia, Lesch-Nyhan syndrome, phenylketonuria, galactosaemia, Von Gierke's disease, diabetes mellitus, cystic fibrosis, acute myocardial infarction, multiple sclerosis and rheumatoid arthritis. Examples where tissue engineering could be applied are those related to cartilage, bone, tendon, ligament, intervertebral disc or muscle substance defects. Whereas state-of-the-art approaches based on random or virus-mediated integration of target DNA into the cell's genome often lead to detrimental effects on cell function with the threat that the cells become tumorigenic (e.g. through the impairment of genes involved in controlling cell cycle progression), the possibility provided by this invention to pre-select favourable integration sites represents a major advantage. Further, as might be desired in certain applications, it is possible to pre-select for integration sites that keep the expression levels in a defined range. From a safety point of view it is of particular importance that, except for the nucleotide sequence comprising the gene(s) of interest, no foreign DNA will remain in the producer cell line. Further, this sequence can be designed such that it does not contain any DNA of viral origin.
3. For the generation of producer cell lines with defined integration sites for more comparable expression studies using different vectors or vector libraries. The different vectors may contain various promoters, enhancers or other control elements affecting transcriptional or translational activity. The vector libraries may be large collections of mutants generated by random mutagenesis.
4. For the integration of vector libraries into an industrial production set-up. An example would be a library generated by random mutagenesis at specific positions within the coding sequence of a signal peptide. It has been shown that certain positions have a substantial effect on the efficiency with which signal peptides modulate protein synthesis and secretion (Zhang et al. 2005). Producer cell lines being homogeneous with respect to the integration site could readily be screened for the best performing subclone in a population being heterogeneous with respect to the library.
5. For the differential production of individual subunits of complex proteins where it is advantageous/required to provide the subunits in non-stoichiometric amounts. An example is an antibody where under normal conditions, e.g. in plasma cells, an excess of light chain with respect to the amount of heavy chain is produced. While the invention describes targeting transgenes into a single locus of the cell's genome, a future direction could involve several targeted loci within the same cell with differential transcriptional activity. They can be isolated by subjecting the core cell line to further rounds of transfection and subsequent sorting by using additional plasma membrane protein marker(s) and site-specific recombination systems.

In this invention, at least two vectors may be used, one containing the first nucleotide sequence for generating the core cell line(s) (see FIGS. 2A,C,E as examples) and the other containing the second nucleotide sequence for generating the producer cell line(s) (see FIG. 2B,D,F as examples). Regarding the components assembled to constitute the two nucleotide sequences examples of possible modifications are listed below.
1. Instead of containing site-specific recombination sites at their 5′ and 3′ ends, the first and second nucleotide sequences could contain pair-wise identical sequence regions at their ends allowing for homologous recombination.
2. In addition to encoding the plasma membrane protein and, optionally, drug resistance markers, the first nucleotide sequence could encode another marker that can be recognised by cell screening technology. This could be desirable in cases where the protein of interest is an intracellular protein and cells with optimal chromosomal integration sites are to be identified based on the screening for appropriate expression of a protein marker also remaining within the cell. Candidate markers would be fluorescent proteins (e.g. eGFP) or β-lactamase.
3. Instead of using transcriptional and translational control elements present on a commercially available vector, in the second nucleotide sequence these elements can be replaced by ones having been identified as being particularly efficient in modulating synthesis/secretion of a protein of interest. Examples are selected 5′UTRs, 3′UTRs and signal peptide coding regions and combinations thereof (Knappskog et al. 2007).

Related to the invention, appropriate nucleotide sequences, vectors, core cell lines and protocols required to generate producer cell lines for particular proteins of interest may be included in kits for commercial or research applications. Such a kit could contain, for example, the following components:
(i) An isolated genetically modified eukaryotic cell(s) comprising a first nucleotide sequence as defined above and at least one fourth nucleotide which comprises a 5′ nucleotide sequence and a 3′ nucleotide sequence, wherein said 5′ and 3′ nucleotide sequences are compatible with those present in and flanking the first nucleotide sequence. Said fourth nucleotide sequence is identical to said second nucleotide sequence except for not comprising the coding sequence(s) of the protein(s) of interest. It may be contained in a vector.
(ii) One or more toxic and/or protective agents to be used for the selection.
(iii) A third nucleotide sequence(s) encoding a recombinase(s).
(iv) All required protocols related to this invention.

### EXAMPLES

The following examples are intended to illustrate the invention, but not to limit the invention in any manner, shape, or form, either explicitly or implicitly.

### Example 1

Proof-of-concept using α_{M}β₂ integrin (CD11b/CD18) as toxin receptor and Adenylate cyclase toxin (CyaA) as toxic agent

### Step 1: Verification of the Stringency of Selection

### First Nucleotide Sequence

The expression vector pcDNA-select1, a derivative of pcDNA 3.1(+) (Invitrogen), was generated, harbouring the genes for the plasma membrane protein flanked by the recombination sites for gene exchange (vector map: FIG. 2A). As a plasma membrane protein the integrin protein CD18/CD11b was chosen. The gene encoding the CD18 subunit (Accession number NM_{—}008404) was copied and amplified using the genome of mouse cell line J774A.1 as template, whereas the gene for the CD11b subunit (Accession number NM_{—}008401) was ordered from a company providing DNA synthesis services (GeneArt). The site-specific recombination sites chosen were the non-interacting heterospecific FRTwt/FRT-5F sites described by Ellermeier et al. (2002) and Schucht et al. (2006). The pcDNA-select 1 vector was used for transfection in its entirety, thus constituting the first nucleotide sequence.

### Generation of Stably Transfected Cells

CHO-S cells (Invitrogen) were grown and propagated according to the manufacturer's recommendations. Only cells from cultures with a viability of greater than 95% were used for transfection, which was performed with the Amaxa nucleofection system (Amaxa). For each transfection a total of 10⁷ viable cells and a DNA amount of the first nucleotide sequence (pcDNA-select1) of 20 μg were used. Immediately after the electroporation procedure, cells were transferred to 6-well plates containing growth medium pre-warmed to 37° C. After two hours of incubation at 37° C. and 7% CO₂ for recovery, cells were transferred to larger culture flasks. Selection for cells with stably integrated DNA was started 48 h post-transfection by applying 400 μg/ml Hygromycin B (InvivoGen) to the cell culture medium.

In order to establish a stably transfected pool of cells expressing the toxin receptor, after one week of selection the cells were subjected to fluorescence-activated cell sorting (FACS). Cells were stained for CD11b/CD18 expression with antibody rat-anti-mouse-CD11b-Fluorescein Isothiocyanate conjugate (AbD Serotec) and rat-anti-mouse-CD18-R-Phycoerythrin conjugate (AbD Serotec), respectively, diluted 1:50. After incubation for 30 min in the dark at room temperature, cells were washed with PBS to remove unbound staining antibody, resuspended in 400 μl PBS with 0.1 mg/ml Propidium Iodide and transferred to FACS sample tubes. Cell sorting for high level expression of the receptor proteins was performed with a BD FACS Vantage flow cytometer (Becton Dickinson). A 5W Argon Laser (Coherent), tuned to 488 nm, with 100 mW output power was used. Sorted cells were pooled in a culture flask and cultivated under selection pressure with Hygromycin B (400 μg/ml cell culture). Subsequently, monitoring of stable CD18/CD11b receptor expression was carried out once a week by flow cytometry as described above. Bulk enrichment of positive CHO cells was performed by flow cytometry cell sorting as described above.

The generated cell pool represents the “core cell line”, harbouring the receptor-encoding genes flanked by recombination sites integrated in the cells' genome.

### Toxicity Test

In order to verify the stringency of the toxin-sensitivity based selection, a sample of the receptor-positive CHO-S cells was mixed with a sample of non-transfected receptor-negative CHO-S cells and the mixed population observed during 48 hours after addition of the toxic agent to the medium.

A total of 3×10⁵ receptor-positive cells and the same number of receptor-negative cells were incubated in 3 ml CD-CHO medium (Invitrogen) in a 6-well plate at 37° C. and 7% CO₂ after addition of Adenylate cyclase toxin (List Biological Laboratories) at a concentration of 0.5 μg/ml cell culture.

A 0.5 ml sample was taken after 48 h, centrifuged at 190 g for 10 min and washed twice with PBS at room temperature. Cell pellets were resuspended in 100 μl PBS+20% FCS containing the staining antibodies rat-anti-mouse-CD11b-Fluorescein Isothiocyanate conjugate (AbD Serotec) and rat-anti-mouse-CD18-R-Phycoerythrin conjugate (AbD Serotec) diluted 1:50. After incubation for 30 min in the dark at room temperature, cells were washed with PBS again to remove unbound staining antibody, resuspended in 400 μl PBS with 0.1 mg/ml Propidium Iodide and transferred to FACS sample tubes. Samples were analyzed on a BD FACS Vantage flow cytometer (Becton Dickinson). A non-expressing CD11b and/or CD18 receptors CHO cell line was used as a negative control to adjust the fluorescence signal to lie between 0 and 10 on the logarithmic scales for Fl 2 (R-PE). All other samples were analyzed using the same instrument settings. Cells were differentiated based on whether or not they expressed the CD11b/CD18 proteins, viability was measured on Fl3 based on exclusion of Propidium Iodide.

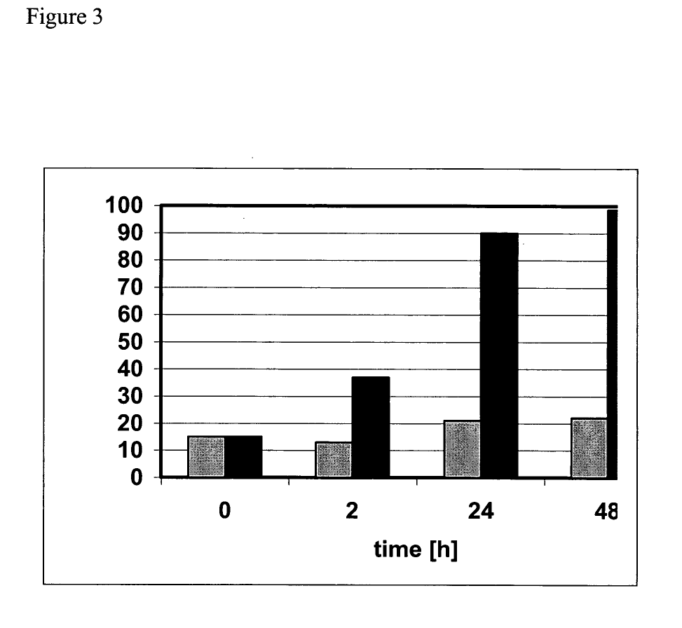

The result was extremely convincing: almost all of the receptor-positive cells died within this short time period, whereas the receptor-negative cells were not affected (FIG. 3). The fast and stringent toxin response of the receptor-positive cells is the prerequisite that in a mixed cell population of receptor-positive cells and cells expressing the protein of interest, only the latter will survive after toxin addition.

### Step 2: Verification of the Efficiency of Gene Exchange

### Second Nucleotide Sequence

The vector pcDNA-target1 was generated by exchanging the fragment within the FRTwt/FRT-5F recombination sites in vector pcDNA-select1 (see FIG. 2A) with a multiple cloning site generated by PCR, which then was used to insert the coding sequences for an immunoglobulin G1 (IgG1) light chain (LC) and IgG1 heavy chain (HC) and the corresponding regulatory elements (vector map: FIG. 2B). The pcDNA-target1 vector was used for transfection in its entirety, thus constituting the second nucleotide sequence.

### Recombinase-Encoding Vector

The gene for the FlpE recombinase (Ellermeier et al. 2002; Schucht et al. 2006) was ordered from GeneArt and inserted into the multiple cloning site of the vector pcDNA3.1(+) (Invitrogen), generating the vector pcDNA-FlpE (vector map not shown).

### Co-Transfection and Gene Exchange

In order to convert the CHO-S cell pool stably expressing the CD11b/CD18 receptor (representing a “core cell line”) into a pool expressing IgG1 (and thus a “producer cell line”), the respective genes were now to be exchanged in a site-specific recombination reaction. The temporarily expressed recombinase encoded by the vector pcDNA-FlpE recognises the recombination sites flanking both the CD18/CD11b genes integrated in the cells' genome and the HC/LC genes contained in the incoming vector pcDNA-target1.

Co-transfections of pcDNA-FlpE and pcDNA-target1 were carried out using the Amaxa nucleofection system. Only cells from cultures with a viability of greater than 95% were subjected to transfection. For each co-transfection a total of 10⁷ viable cells and DNA amounts of 20 μg pcDNA-FlpE DNA and 6 μg pcDNA-target DNA were used, thus following the recommendations by Wirth and Hauser (2004) to apply a DNA ratio of approx. 3:1. Immediately after the electroporation procedure, cells were transferred to E-well plates containing growth medium pre-warmed to 37° C. After two hours of incubation at 37° C. and 7% CO₂ for recovery, cells were transferred to larger culture flasks.

After the transfection, the cells were propagated by routine serial passage for 3 weeks, allowing for a turnover of the receptor protein, such that cells now expressing antibody would have lost the receptor protein on their surface. Further, this extended time period would ensure that any non-integrated HC/LC recombination vector is lost during cell division, and that any antibody initially expressed by the non-integrated HC/LC recombination vector is removed by exchange of growth medium during cell passaging.

### Monitoring Gene Exchange

After gene exchange, cells were analysed for antibody production with two different methods. Firstly, samples of about 10⁶ cells were taken 3 weeks after gene exchange and HC titers in the growth medium determined by enzyme-linked immuno sorbent assay (ELISA). MaxiSorp plates (Nunc) were coated with goat-anti-human-gamma-chain antibody overnight at 4° C. Culture supernatant was applied to the plate in two-fold serial dilutions together with an appropriate antibody standard at a concentration of 200 ng/ml. Bound antibody was detected with the respective antibody used for coating conjugated to horseradish peroxidase and o-Phenylenediamine Dihydrochloride (OPD) as substrate.

Secondly, antibody production was monitored using Immunofluorescence analysis to determine the intracellular content of antibody. For this, samples of about 10⁶ cells were taken 3 weeks after gene exchange. Cells were fixed in 70% ethanol and stored at 4° C. until staining for intracellular product content. Fixed cells were washed twice with Tris buffer (100 mM Tris, 2 mM MgCl₂, 0.1% Triton X-100, pH 7.4) followed by one wash with Tris buffer containing 20% FCS. Cells were then resuspended in 200 μl Tris buffer with FCS containing fluorescently labeled staining antibodies diluted 1:50. As staining antibody rat-anti-mouse-CD11b-Fluorescein Isothiocyanate conjugate (AbD Serotec) and rat-anti-mouse-CD18-R-Phycoerythrin conjugate (AbD Serotec) were used. After incubation for one hour at 37° C. in the dark samples were washed once with Tris buffer. Pellets were resuspended in Tris buffer and were transferred to FACS sample tubes. Samples were analyzed on a BD FACSCalibur™ flow cytometer (Becton Dickinson). A non-transfected CHO-S cell line was used as a negative control to set the fluorescence signals for Fl 1 (FITC) and Fl 2 (R-PE) to lie between 0 and 10 on the logarithmic scales. Compensation of spectral overlap between the FITC signal and the R-PE signal was adjusted using positive cells stained with each antibody separately.

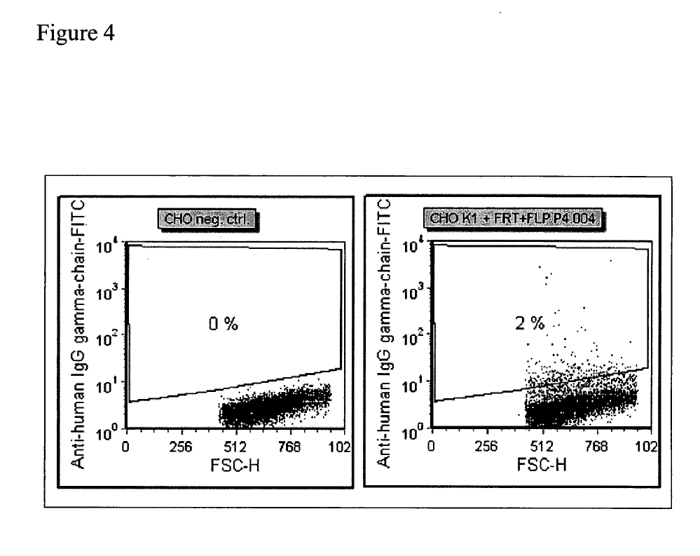

The ELISA analysis measured a concentration of 0.1 μg antibody/ml medium and the flow cytometry analysis showed that 2% of cells contained antibody, as seen from the image in FIG. 4. This result was achieved without selection of cells that had performed gene exchange, thus allowing the estimation that the efficiency of gene exchange was approximately 2%. In addition, the approximate cell specific productivity of the cells after gene exchange can be calculated as follows: if a mixed cell population (expressing both receptor and antibody) containing 2% antibody-expressing cells has an antibody titer of 0.1 μg/ml, a pure population (expressing only antibody) would have a titer of 5 μg/ml, which, under the conditions used, is already in the range of a normal production cell line, although no optimisation has yet been performed. The pure population thus can be readily generated from the mixed population through addition of toxin, then representing the “producer cell line” for the antibody chosen.

Considering the various parameters which can be optimised in order to generate high-producer “core cell lines” with homogeneous receptor-expression levels and stable genomic integration sites, the tremendous potential of the selection system in an industrial setting has become evident.

### Example 2

Proof-of-concept using Guanylyl cyclase C (GC-C) as toxin receptor and Heat-stable enterotoxin (STₐ) as toxic agent.

### Step 1: Verification of the Stringency of Selection

In order to strengthen the proof-of-concept of selection stringency provided with EXAMPLE 1 and to demonstrate its broad validity, the same line of experiments were performed using a different receptor/toxin system.

### First Nucleotide Sequence

The expression vector pcDNA-select2 differs from pcDNA-select 1 (see FIG. 202A) in two respects. It harbours the gene for the plasma membrane protein Guanylyl cyclase C (Hasegawa et al. 2005) instead of the genes for CD18/CD11b, and the receptor-encoding gene is flanked by the recombination sites loxP/lox2272 (Saito and Tanaka) instead of the FRTwt/FRT-5F sites. For the map of pcDNA-select2 see FIG. 2C. The gene encoding Guanylyl cyclase C was ordered from GeneArt. The pcDNA-select2 vector was used for transfection in its entirety, thus constituting the first nucleotide sequence.

### Generation of Stably Transfected Cells

See EXAMPLE 1

### Toxicity Test

See EXAMPLE 1

The results obtained from CHO-S cell pools stably transfected with pcDNA-select2 were similar to those obtained in EXAMPLE 1. Cells were efficiently killed by addition of 0.5 μg/ml of Heat-stable toxin (Sigma-Aldrich) within 48 h, while non-transfected CHO-S cells were not affected by the toxin at all up to concentrations of 2 μg/ml.

### Step 1: Verification of Efficient Gene Exchange

In order to strengthen the proof-of-concept of gene exchange efficiency provided with EXAMPLE 1 and to demonstrate its broad validity, the same line of experiments were performed using a different site-specific recombination system.

### Second Nucleotide Sequence

The vector pcDNA-target2 was generated by exchanging the fragment within the loxP/lox2272 recombination sites in vector pcDNA-select2 (see FIG. 2C) with a multiple cloning site generated by PCR, which then was used to insert the coding sequences for an IgG1 LC and IgG1 HC and the corresponding regulatory elements (vector map: FIG. 2D). The pcDNA-target2 vector was used for transfection in its entirety, thus constituting the second nucleotide sequence.

### Recombinase-Encoding Vector

The gene for the Cre recombinase (Accession number AB363405.1) was ordered from GeneArt and inserted into the multiple cloning site of the vector pcDNA3.1(+) (Invitrogen), generating the vector pcDNA-Cre (vector map not shown).

### Co-Transfection

See EXAMPLE 1

Co-transfections were performed with pcDNA-FlpE and pcDNA-target2 and the CHO-S cell pool stably expressing the GC-C receptor converted into a pool expressing IgG1.

### Monitoring Gene Exchange

See EXAMPLE 1

The results achieved were comparable with those obtained in EXAMPLE 1.

### Example 3

### Generating a Producer Cell Line from a Core Cell Line

### Step 1: Generation of a Core Cell Line

### First Nucleotide Sequence

The expression vector pUTR-select (vector map: FIG. 2E) harbouring the coding sequences for the plasma membrane proteins CD11b (Accession number NM_{—}008401) and CD18 (Accession number (NM_{—}008404) flanked by the FRTwt/FRT-5F recombination sites (Ellermeier et al. 2002; Schucht et al. 2006) was cut with restriction endonucleases NruI and PmeI and the DNA fragments separated by electrophoresis on an ethidium bromide stained 0.7% (w/v) agarose gel. The 11 kbp DNA fragment constituting the first nucleotide sequence was excised and purified using the EZNA MicroElute Gel Extraction Kit (Omega Bio-Tek) following the manufacturer's instructions and used for transfection.

### Generation of Stably Transfected Cells

CHO-K1 cells (ATTC) adapted to grow in protein-free medium were propagated according to the manufacturer's recommendations. 10⁷ cells were transfected by electroporation/nucleofection (Amaxa) following the manufacturer's instructions. Selection for cells with stably integrated DNA was started 24 h post-transfection by applying 400 μg/ml Hygromycin B (InvivoGen) to the cell culture medium for two weeks. For more details see EXAMPLE 1.

### Fluorescent-Activated Cell Sorting (FACS)

Cells expressing both receptor subunits at high levels were stained using the appropriate antibodies and sorted into 96 well plates at one cell per well. Cells were expanded and cultivated in the absence of selection marker for at least three months. During this time the maintenance of receptor expression levels was confirmed by FACS. For more details see EXAMPLE 1.

Southern blot was used to determine the number of integrated nucleotide copies, fluorescence in situ hybridization (FISH) was used to identify the chromosomal integration site of the toxin receptor genes. Subclones with different expression levels of the two membrane proteins that were proven to be stable during that time were selected as “core cell lines” for different applications requiring different expression levels. Southern blot and FISH analyses were done according to standard procedures.

### Step 2: Generation of a Producer Cell Line

### Second Nucleotide Sequence

The vector pUTR-target harbouring the coding sequences for an IgG1 LC and IgG1 HC (vector map: FIG. 2F) flanked by the FRTwt/FRT-5F recombination sites (Ellermeier et al. 2002; Schucht et al. 2006) was digested with the restriction enzymes NruI and PmeI and the DNA fragments separated by electrophoresis on an ethidium bromide stained 0.7% (w/v) agarose gel. The 7.4 kbp DNA fragment constituting the second nucleotide sequence was excised from the gel and purified using the EZNA MicroElute Gel Extraction Kit following the manufacturer's recommendations.

### Recombinase Encoding Sequence

See EXAMPLE 1

### Co-Transfection

The pcDNA-FlpE vector and second nucleotide sequence DNAs were mixed at a 3:1 molar ratio and transfected into the core cell line cells using electroporation/nucleofection (Amaxa). To remove cells that had not exchanged the gene cassette, Adenylate cyclase toxin (Sigma-Aldrich) was applied at 5 μg/ml to the culture medium for three days starting 72 h after transfection. For more details see EXAMPLE 1.

### FACS Single Cell Sorting

Surviving cells were sorted for high levels of secretion of the antibody encoded by the second nucleotide sequence. This was performed using a single cell secretion assay and the cells were sorted into 96 well plates at one cell per well. Cell specific production rates were assessed by ELISA. Southern blot and quantitative polymerase chain reaction (PCR) were used to confirm absence of plasma membrane receptor genes and presence of antibody genes. For more details see EXAMPLE 1. Quantitative PCR was done according to standard procedure.

### REFERENCES

Alt, F. W., Kellems, R. E., Bertino, J. R. and Schimke, R. T. (1978). “Selective multiplication of dihydrofolate reductase genes in methotrexate-resistant variants of cultured murine cells.” J Biol Chem 253(5): 1357-70.
Borth, N., Zeyda, M., Kunert, R. and Katinger, H. (2000). “Efficient selection of high-producing subclones during gene amplification of recombinant Chinese hamster ovary cells by flow cytometry and cell sorting.” Biotechnol Bioeng 71(4): 266-73.
Browne, S. M. and Al-Rubeai, M. (2007). “Selection methods for high-producing mammalian cell lines.” Trends Biotechnol 25(9): 425-32.
Carroll, S, and Al-Rubeai, M. (2004). “The selection of high-producing cell lines using flow cytometry and cell sorting.” Expert Opin Biol Ther 4(11): 1821-9.
Cockett, M. I., Bebbington, C. R. and Yarranton, G. T. (1990). “High level expression of tissue inhibitor of metalloproteinases in Chinese hamster ovary cells using glutamine synthetase gene amplification.” Biotechnology (N Y) 8(7): 662-7.
Coroadinha, A. S., Schucht, R., Gama-Norton, L., Wirth, D., Hauser, H. and Carrondo, M. J. (2006). “The use of recombinase mediated cassette exchange in retroviral vector producer cell lines: predictability and efficiency by transgene exchange.” J Biotechnol 124(2): 457-68.
El-Azami-El-Idrissi, M., Bauche, C., Loucka, J., Osicka, R., Sebo, P., Ladant, D. and Leclerc, C. (2003). “Interaction of Bordetella pertussis adenylate cyclase with CD11b/CD18: Role of toxin acylation and identification of the main integrin interaction domain.” J Biol Chem 278(40): 38514-21.
Ellermeier, C. D., Janakiraman, A. and Slauch, J. M. (2002). “Construction of targeted single copy lac fusions using lambda Red and FLP-mediated site-specific recombination in bacteria.” Gene 290(1-2): 153-61.
Hasegawa, M. and Shimonishi, Y. (2005). “Recognition and signal transduction mechanism of Escherichia coli heat-stable enterotoxin and its receptor, guanylate cyclase C.” J Pept Res 65(2): 261-71.
Hoess, R. H., Wierzbicki, A. and Abremski, K. (1986). “The role of the loxP spacer region in P1 site-specific recombination.” Nucleic Acids Res 14(5): 2287-300.
Joubert, O., Voegelin, J., Guillet, V., Tranier, S., Werner, S., Colin, D. A., Serra, M. D., Keller, D., Monteil, H., Mourey, L. and Prevost, G. (2007). “Distinction between Pore Assembly by Staphylococcal alpha-Toxin versus Leukotoxins.” J Biomed Biotechnol 2007: 25935.
Knappskog, S., Ravneberg, H., Gjerdrum, C., Trosse, C., Stem, B. and Pryme, I. F. (2007). “The level of synthesis and secretion of Gaussia princeps luciferase in transfected CHO cells is heavily dependent on the choice of signal peptide.” J Biotechnol 128(4): 705-15.
Kwaks, T. H. and Otte, A. P. (2006). “Employing epigenetics to augment the expression of therapeutic proteins in mammalian cells.” Trends Biotechnol 24(3): 137-42.
Langer, S. J., Ghafoori, A. P., Byrd, M. and Leinwand, L. (2002). “A genetic screen identifies novel non-compatible loxP sites.” Nucleic Acids Res 30(14): 3067-77.
Lauth, M., Spreafico, F., Dethleffsen, K. and Meyer, M. (2002). “Stable and efficient cassette exchange under non-selectable conditions by combined use of two site-specific recombinases.” Nucleic Acids Res 30(21): e115.
Mattanovich, D. and Borth, N. (2006). “Applications of cell sorting in biotechnology.” Microb Cell Fact 5: 12.
Nakayama, M., Hisatsune, J., Yamasaki, E., Nishi, Y., Wada, A., Kurazono, H., Sap, J., Yahiro, K., Moss, J. and Hirayama, T. (2006). “Clustering of Helicobacter pylori VacA in lipid rafts, mediated by its receptor, receptor-like protein tyrosine phosphatase beta, is required for intoxication in AZ-521 Cells.” Infect Immun 74(12): 6571-80.
Saito, I. and Tanaka, K. “Patent CA2309386.”
Santos, A., San Mauro, M., Abrusci, C. and Marquina, D. (2007). “Cwp2p, the plasma membrane receptor for Pichia membranifaciens killer toxin.” Mol Microbiol 64(3): 831-43.
Schlake, T. and Bode, J. (1994). “Use of mutated FLP recognition target (FRT) sites for the exchange of expression cassettes at defined chromosomal loci.” Biochemistry 33(43): 12746-51.
Schucht, R., Coroadinha, A. S., Zanta-Boussif, M. A., Verhoeyen, E., Carrondo, M. J. T., Hauser, H. and Wirth D. (2006). “A new generation of retroviral producer cells: predictable and stable virus production by Flp-mediated site-specific integration of retroviral vectors.” Mol Ther 14(2): 285-92.
Thyagarajan, B., Olivares, E. C., Hollis, R. P., Ginsburg, D. S, and Calos, M. P. (2001). “Site-specific genomic integration in mammalian cells mediated by phage phiC31 integrase.” Mol Cell Biol 21(12): 3926-34.
Wirth, D., Gama-Norton, L., Riemer, P., Sandhu, U., Schucht, R. and Hauser, H. (2007). “Road to precision: recombinase-based targeting technologies for genome engineering.” Curr Opin Biotechnol 18(5): 411-9.
Wirth, D. and Hauser, H. (2004). “Flp-mediated integration of expression cassettes into FRT-tagged chromosomal loci in mammalian cells.” Methods Mol Biol 267: 467-76.
Wong, E. T., Kolman, J. L., Li, Y. C., Mesner, L. D., Hillen, W., Berens, C. and Wahl, G. M. (2005). “Reproducible doxycycline-inducible transgene expression at specific loci generated by Cre-recombinase mediated cassette exchange.” Nucleic Acids Res 33(17): e147.
Wurm, F. M. (2004). “Production of recombinant protein therapeutics in cultivated mammalian cells.” Nat Biotechnol 22(11): 1393-8.
Zhang, L., Leng, Q. and Mixson, A. J. (2005). “Alteration in the IL-2 signal peptide affects secretion of proteins in vitro and in vivo.” J Gene Med 7(3): 354-65.

1. A method for isolation of a genetically modified eukaryotic cell(s) comprising the steps of
a) providing a eukaryotic cell(s) and at least one first nucleotide sequence, wherein said first nucleotide sequence comprises a 5′ nucleotide sequence and at least one gene encoding a plasma membrane protein and a 3′ nucleotide sequence, wherein said 5′ and 3′ nucleotide sequences allow for the exchange of the enclosed nucleotide sequence,
b) transfecting said first nucleotide sequence into the genome of said eukaryotic cell(s),
c) screening for a core cell(s) wherein said core cell(s) has at least one first nucleotide sequence integrated in the genome and expressing said at least one gene encoding a plasma membrane protein, wherein said plasma membrane protein is a toxic protein or a toxin receptor,
d) propagating said core cell(s) and
e) obtaining at least one core cell line.

2. The method according to claim 1, wherein said method comprises the steps of
a) providing at least one second nucleotide sequence which comprises a 5′ nucleotide sequence and at least one gene encoding a protein(s) of interest and a 3′ nucleotide sequence, wherein said 5′ and 3′ nucleotide sequences are compatible with those present in and flanking the first nucleotide sequence,
b) replacing said first nucleotide sequence in said core cell line(s) with said second nucleotide sequence,
c) selecting for at least one producer cell wherein said producer cell(s) has at least one second nucleotide sequence integrated into the genome and expressing said at least one gene encoding a protein(s) of interest,
d) propagating said producer cell(s) and
e) obtaining at least one producer cell line.

3. The method according to claim 1, wherein said plasma membrane protein mediates cell death after application of a toxic agent and/or removal of a protective agent.

4. The method according to claim 3, wherein said plasma membrane protein is selected from the group listed in Table 1.

5. The method according to claim 4, wherein said plasma membrane protein is α_{M}β₂ integrin and said toxic agent is adenylate cyclise toxin.

6. The method according to claim 1, wherein said 5′ and 3′ nucleotide sequences, allowing for the exchange of the enclosed nucleotide sequence, are target sites for a recombinase(s).

7. The method according to claim 1, wherein said 5′ and 3′ nucleotide sequences, allowing for the exchange of the enclosed nucleotide sequence, are heterospecific recombination sites.

8. The method according to claim 7, wherein said heterospecific recombination sites are selected from the group listed in Table 2.

9. The method according to claim 1, wherein said recombinase(s) is encoded by a third nucleotide sequence and/or endogenously expressed by said eukaryotic cell(s).

10. The method according to claim 1, wherein said eukaryotic cell is an animal cell, plant cell, fungal cell or yeast cell.

11. The method according to claim 10, wherein said animal cell is a mammalian cell.

12. The method according to claim 11, wherein said mammalian cell is selected from the group consisting of primate-, monkey- and rodent-derived cells.

13. The method according to claim 12, wherein said primate cell is of Homo sapiens or Pan troglodytes origin, said monkey cell is of Cercopithecus aethiops origin and said rodent cell is of Cricetulus griseus, Mesocricetus auratus, Rattus norvegicus, Oryctolagus cuniculus or Mus musculus origin.

14. The method according to claim 10, wherein said eukaryotic cell belongs to any of the cell line families CHO, NSO, 293, myeloma, NOS, COS, BHK, HeLa and PER.C6.

15. The method according to claim 1, wherein said first nucleotide sequence comprises at least one gene encoding a drug resistance marker(s).

16. The method according to claim 15, wherein said at least one gene encoding a drug resistance marker(s) is an antibiotic resistance gene.

17. The method according to claim 16, wherein said antibiotic resistance gene is selected from the group comprising neomycin phosphotransferase I, neomycin phosphotransferase II, hygromycin B phosphotransferase, blasticidin, blasticidin S deaminase, puromycin N-acetyl-transferase and bleomycin resistance gene.

18. The method according to any of preceding claims claim 1, wherein said screening is done by dilution cloning, flow cytometry, cell sorting, magnetic cell sorting or automated systems.

19. A first nucleotide sequence wherein said first nucleotide sequence comprises a 5′ nucleotide sequence and at least one gene encoding a plasma membrane protein and a 3′ nucleotide sequence, wherein said 5′ and 3′ nucleotide sequences allow for the exchange of the enclosed nucleotide sequence and wherein said plasma membrane protein is a toxic protein or a toxin receptor.

20. The nucleotide sequence according to claim 19, wherein said plasma membrane protein mediates cell death after application of a toxic agent and/or removal of a protective agent.

21. The nucleotide sequence according to claim 20, wherein said plasma membrane protein is selected from the group listed in Table 1.

22. The nucleotide sequence according to claim 21, wherein said plasma membrane protein is α_{M}β₂ integrin and said toxic agent is adenylate cyclise toxin.

23. The nucleotide sequence according to claim 19, wherein said 5′ and 3′ nucleotide sequences, allowing for the exchange of the enclosed nucleotide sequence, are target sites for a recombinase(s).

24. The nucleotide sequence according to claim 23, wherein said 5′ and 3′ nucleotide sequences, allowing for the exchange of the enclosed nucleotide sequence, are heterospecific recombination sites.

25. The nucleotide sequence according to claim 24, wherein said heterospecific recombination sites are selected from the group listed in Table 2.

26. A vector comprising the nucleotide sequence according to claim 19.

27. An isolated genetically modified eukaryotic cell(s) comprising a first nucleotide sequence according to claim 19.

28. An isolated genetically modified eukaryotic cell(s) obtainable by the method according to claim 1.

29. A kit comprising an isolated genetically modified eukaryotic cell(s) comprising a first nucleotide sequence according to claim 19 and at least one fourth nucleotide sequence which comprises a 5′ nucleotide sequence and a 3′ nucleotide sequence, wherein said 5′ and 3′ nucleotide sequences are compatible to those present in the first nucleotide sequence.

30. The kit according to claim 29, which further comprises a toxic and/or protective agent(s).

31. The kit according to claim 30, wherein said toxic and/or protective agent(s) is selected from the group listed in Table 1.

32. The kit according to claim 29, wherein said plasma membrane protein is am % integrin and said toxic agent is adenylate cyclise toxin.

33. The kit according to claim 29, which further comprises a nucleotide sequence encoding a recombinase(s).

34. The process of using the method according to claim 1, said nucleotide sequence, said vector, said isolated genetically modified eukaryotic cell(s) and said kit.
